(19) United States
(12) Patent Application Publication
(10) Pub. No.: US 2025/0017743 A1
(43) Pub. Date: Jan. 16, 2025

(54) ANCHOR AND INTERBODY FOR INTERBODY FUSION

(71) Applicant: Evolution Spine, LLC, Dallas, TX (US)

(72) Inventors: Keith Alan Roby, Allen, TX (US); Todd Wallenstein, Ashburn, VA (US); Benjamin Keller, Austin, TX (US); James Mirda, Germantown, TN (US); Francia Sanchez, Knoxville, TN (US)

(21) Appl. No.: 18/770,014

(22) Filed: Jul. 11, 2024

Related U.S. Application Data
(60) Provisional application No. 63/513,203, filed on Jul. 12, 2023.

(51) Int. Cl. A61F 2/44; A61F 2/30; A61F 2/46

(52) U.S. Cl. CPC A61F 2/4465; A61F 2002/30316; A61F 2002/30841; A61F 2002/4625

(57) ABSTRACT

Provided herein are anchors and interbodies for interbody fusion, comprising an interbody for insertion between vertebrae of a human spine, comprising an interbody cage comprising a plurality of anchor receptacles, each anchor receptacle configured to receive an anchor to secure the interbody to a vertebra; and the anchor, comprising a head configured to engage one of the plurality of anchor receptacles; a curved partial-tubular shank connected to or integral with the head at a proximal end of the curved partial-tubular shank, extending from the head, and comprising a plurality of concentric grooves on an exterior surface of the curved partial-tubular shank; and a straight tapered tip portion at a distal end of the curved partial-tubular shank and extending from the curved partial-tubular shank.

### CROSS-REFERENCE TO RELATED APPLICATIONS

This application claims priority to U.S. Provisional Application Ser. No. 63/513,203, filed Jul. 12, 2023, the entire contents of which are incorporated herein by reference.

### STATEMENT OF FEDERALLY FUNDED RESEARCH Not applicable.

### TECHNICAL FIELD OF THE INVENTION

The present invention relates in general to surgery on the human spine, and more particularly, to an anchor and an interbody for use in interbody fusion.

### BACKGROUND OF THE INVENTION

Without limiting the scope of the invention, its background is described in connection with a spinal implant as an example.

An intervertebral disc in a human spine may be replaced by a spinal implant to promote fusion of the vertebrae adjacent to the disc.

U.S. Pat. No. 11,273,048, to Cain et al., is said to disclose an orthopedic implant which generally includes a frame structure and a porous structure, in which both the frame and porous structure at least partially define at least six surfaces which make a three-dimensional profile of the implant, and in which the porous structure is positioned at least partially within the three-dimensional profile.

U.S. Pat. App. Pub. No. 2022/0117753, by Rucker et al., is said to disclose a surgical implant device, including: a solid surface; and a lattice structure disposed adjacent to the solid surface, wherein the lattice structure includes a first plurality of struts that define a first plurality of voids adjacent to the solid surface and a second plurality of struts that define a second plurality of voids remote from the solid surface. Each of the first plurality of struts is said to have an average cross-sectional diameter that is smaller than an average cross-sectional diameter of each of the second plurality of struts. Each of the first plurality of voids is said to have an average internal diameter that is smaller than an average internal diameter of each of the second plurality of voids. The surgical implant device is also said to include a needle-populated porous surface disposed adjacent to the solid surface opposite the lattice structure.

Despite these advances, a need remains for ALIF anchors and interbodies that address the shortcomings discussed herein.

### SUMMARY OF THE INVENTION

As embodied and broadly described herein, an aspect of the present disclosure relates to an anchor and interbody for interbody fusion, comprising: an interbody for insertion between vertebrae of a human spine, comprising: an interbody cage comprising a plurality of anchor receptacles, each anchor receptacle configured to receive an anchor to secure the interbody to a vertebra; and the anchor, comprising: a head configured to engage one of the plurality of anchor receptacles; a curved partial-tubular shank connected to or integral with the head at a proximal end of the curved partial-tubular shank, extending from the head, and comprising a plurality of concentric grooves on an exterior surface of the curved partial-tubular shank; and a straight tapered tip portion at a distal end of the curved partial-tubular shank and extending from the curved partial-tubular shank. In one aspect, the anchor further comprises a plurality of teeth disposed on an edge of the curved partial-tubular shank. In another aspect, the anchor further comprises a plurality of indentations in an exterior surface of the curved partial-tubular shank. In another aspect, the anchor further comprises a plurality of protrusions on an interior surface the curved partial-tubular shank. In another aspect, the anchor further comprises a plurality of grooves in an interior surface the curved partial-tubular shank. In another aspect, the anchor further comprises porosity in an external surface, an internal surface, or both, of the curved partial-tubular shank. In another aspect, an inner diameter and an outer diameter of the curved partial-tubular shank are not concentric, such that the curved partial-tubular shank varies in thickness. In another aspect, the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody. In another aspect, the interbody further comprises an interbody locking tab configured to be moved outward while the head is being inserted into the interbody and to spring inward once the head is fully inserted into the interbody, engaging the head. In another aspect, the straight tapered tip portion comprises one or more reliefs.

As embodied and broadly described herein, an aspect of the present disclosure relates to an anchor and interbody kit for interbody fusion, comprising: an interbody for insertion between vertebrae of a human spine, comprising: an interbody cage comprising a plurality of anchor receptacles, each anchor receptacle configured to receive an anchor to secure the interbody to a vertebra; and the anchor, comprising: a head configured to engage one of the plurality of anchor receptacles; a curved partial-tubular shank connected to or integral with the head at a proximal end of the curved partial-tubular shank, extending from the head, and comprising a plurality of concentric grooves on an exterior surface of the curved partial-tubular shank; and a straight tapered tip portion at a distal end of the curved partial tubular shank and extending from the curved partial-tubular shank; and one or more tools configured to manipulate the interbody or the anchor. In one aspect, the anchor further comprises a plurality of teeth disposed on an edge of the curved partial-tubular shank. In another aspect, the anchor further comprises a plurality of indentations in an exterior surface of the curved partial-tubular shank. In another aspect, the anchor further comprises a plurality of protrusions on an interior surface the curved partial-tubular shank. In another aspect, the anchor further comprises a plurality of grooves in an interior surface the curved partial-tubular shank. In another aspect, the anchor further comprises porosity in an external surface, an internal surface, or both, of the curved partial-tubular shank. In another aspect, an inner diameter and an outer diameter of the curved partial-tubular shank are not concentric, such that the curved partial-tubular shank varies in thickness. In another aspect, the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody. In another aspect, the interbody further comprises an interbody locking tab configured to be moved outward while the head is being inserted into the interbody and to spring inward once the head is fully inserted into the interbody, engaging the head. In another aspect, the straight tapered tip portion comprises one or more reliefs.

As embodied and broadly described herein, an aspect of the present disclosure relates to a method for using an anchor and interbody for interbody fusion, comprising: providing a patient in need of an interbody fusion; providing an anchor and interbody for interbody fusion, comprising: an interbody cage comprising a plurality of anchor receptacles, each anchor receptacle configured to receive an anchor to secure the interbody to a vertebra; and the anchor, comprising: a head configured to engage one of the plurality of anchor receptacles; a curved partial-tubular shank connected to or integral with the head at a proximal end of the curved partial-tubular shank, extending from the head, and comprising a plurality of or concentric grooves on an exterior surface of the curved partial-tubular shank; and a tapered tip portion at a distal end of the curved partial-tubular shank; inserting the interbody between two vertebrae of the patient; and inserting the anchor into one of the plurality of anchor receptacles. In one aspect, the anchor further comprises a plurality of teeth disposed on an edge of the curved partial-tubular shank. In another aspect, the anchor further comprises a plurality of indentations in an exterior surface of the curved partial-tubular shank. In another aspect, the anchor further comprises a plurality of protrusions on an interior surface the curved partial-tubular shank. In another aspect, the anchor further comprises a plurality of grooves in an interior surface the curved partial-tubular shank. In another aspect, the anchor further comprises porosity in an external surface, an internal surface, or both, of the curved partial-tubular shank. In another aspect, an inner diameter and an outer diameter of the curved partial-tubular shank are not concentric, such that the curved partial-tubular shank varies in thickness. In another aspect, the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody. In another aspect, the interbody further comprises an interbody locking tab configured to be moved outward while the head is being inserted into the interbody and to spring inward once the head is fully inserted into the interbody, engaging the head. In another aspect, the straight tapered tip portion comprises one or more reliefs.

### BRIEF DESCRIPTION OF THE DRAWINGS

For a more complete understanding of the features and advantages of the present invention, reference is now made to the detailed description of the invention along with the accompanying figures, in which:

FIGS. 1A-1D show exterior views of an embodiment of an anchor, including circumferential grooves.

FIGS. 2A-2D show exterior views of an embodiment of an anchor, including teeth.

FIGS. 3A-3C show exterior views of an embodiment of an anchor, including spaces on the underside of the anchor.

FIGS. 4A-4B show interior views of an embodiment of an anchor, including interior protrusions.

FIG. 5 shows a cross-section of a head of an embodiment of an anchor.

FIGS. 6A-6C show anchors and an interbody together.

FIGS. 7A-7E show various views of an insertion tool of an embodiment of the invention.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The anchors and spinal implants can be made of biocompatible materials, such as, stainless steel, titanium alloys, aluminum alloys, chromium alloys, metal alloys, CoCrMo, hydroxyapetite, polyether ether ketone (PEEK), polyether ketone (PEKK), carbon fiber, ABS plastic, polyurethane, polyethylene, photo-polymer, resin, fiber-encased resinous material, a polymer, natural materials, other biocompatible materials, and combinations thereof. The anchors and spinal implants may be produced at least in part by traditional subtractive manufacturing, rapid prototyping, 3D printing, stercolithography (STL), selective laser sintering (SLS), fused deposition modeling (FDM), direct metal laser sintering (DMLS), electron beam melting (EBM), multi-jet fusion (MJF), or an additive manufacturing machine.

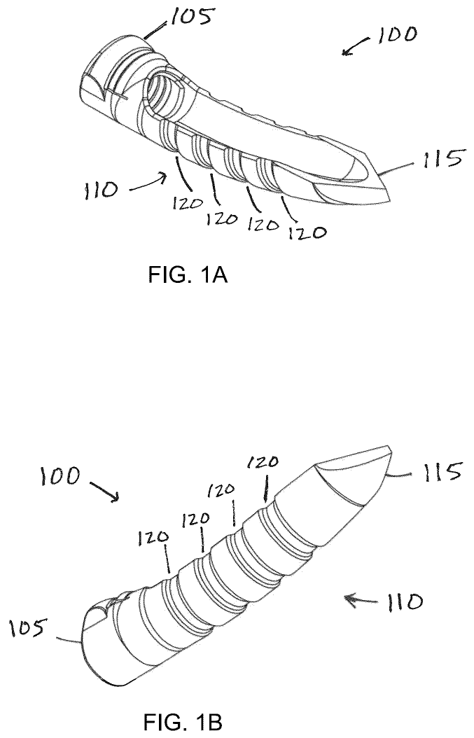

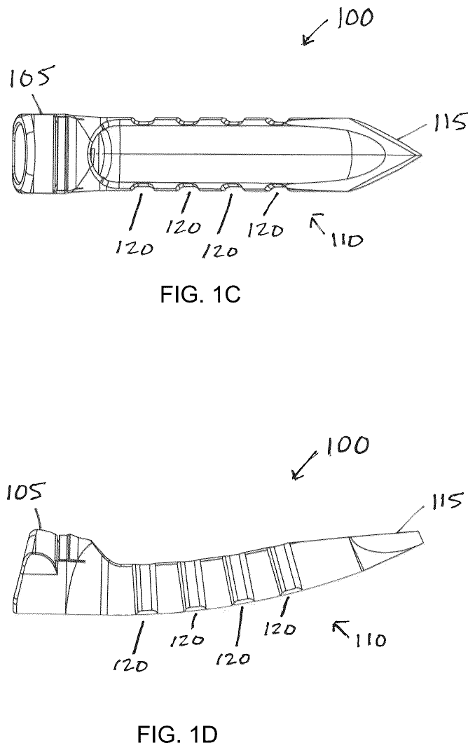

FIGS. 1A-1D show exterior views of an embodiment of an anchor, including circumferential grooves. The anchor 100 includes a head 105, a curved partial-tubular shank 110 extending from the head 105, a straight tapered tip portion 115 extending from the curved partial-tubular shank 110, and a plurality of concentric circumferential grooves 120 in the curved partial-tubular shank 110, into which bone can grow when the anchor 100 is inserted into a vertebra, locking the anchor 100 in place. The plurality of concentric circumferential grooves 120 also provide resistance to motion when the anchor 100 is inserted into a vertebra, tending to hold the anchor in place. The curved partial-tubular shank 110 may have a cylindrical outer profile or a triangular, quadrilateral, or other polygonal outer profile. The straight tapered tip portion 115 may be tapered in a direction of an axis that is distinct from the axis of the straight tapered tip portion 115.

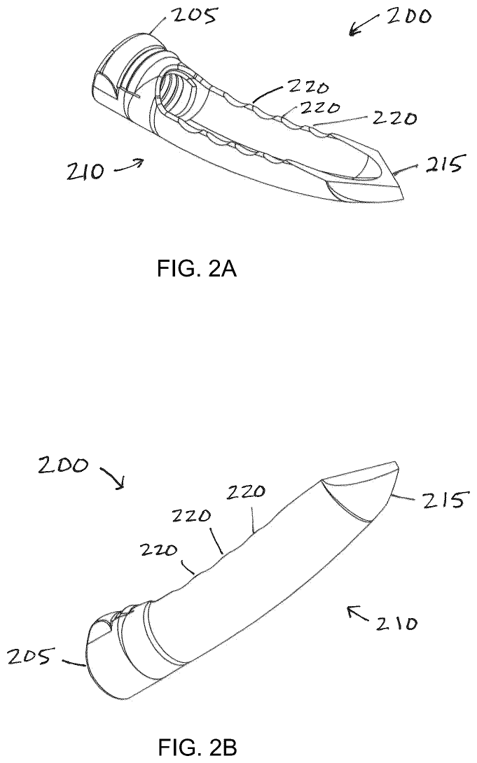

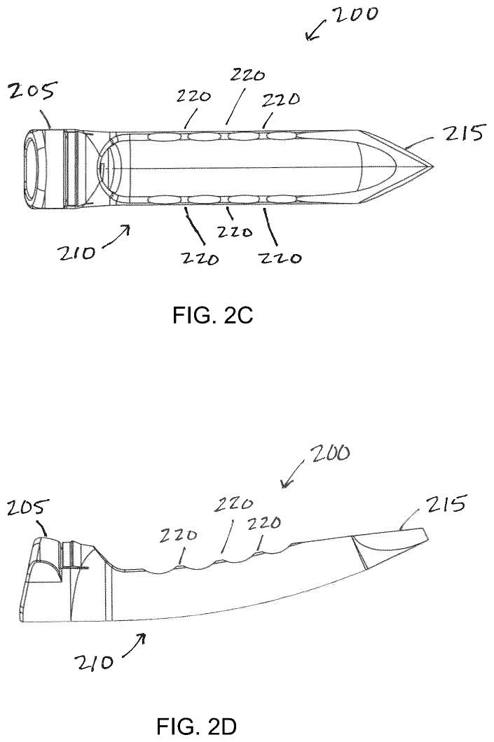

FIGS. 2A-2D show exterior views of an embodiment of an anchor, including teeth. The anchor 200 includes a head 205, a curved partial-tubular shank 210 extending from the head 205, a straight tapered tip portion 215 extending from the curved partial-tubular shank 210, and a plurality of teeth 220 along the two edges of the curved partial-tubular shank 210, which provide resistance to motion when the anchor 200 is inserted into a vertebra, tending to hold the anchor 200 in place. The curved partial-tubular shank 210 may have a cylindrical outer profile or a triangular, quadrilateral, or other polygonal outer profile. The straight tapered tip portion 215 may be tapered in a direction of an axis that is distinct from the axis of the straight tapered tip portion 215.

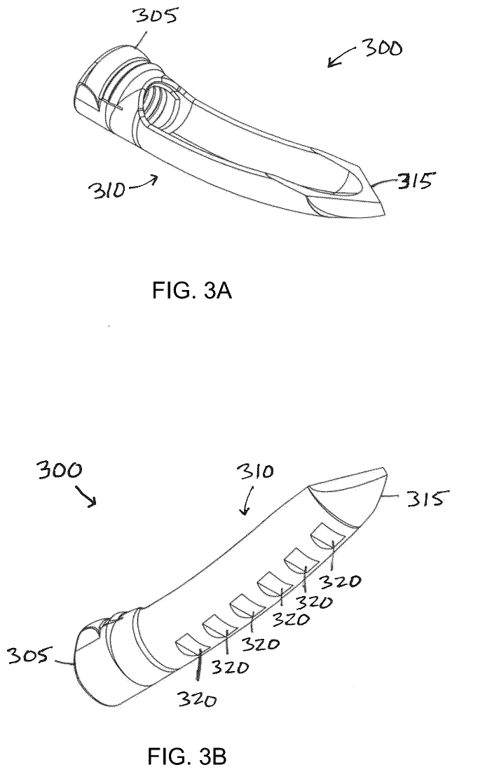

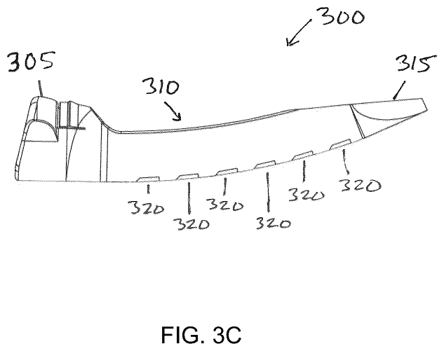

FIGS. 3A-3C show exterior views of an embodiment of an anchor, including indentations on the underside of the anchor. The anchor 300 includes a head 305, a curved partial-tubular shank 310 extending from the head 305, a straight tapered tip portion 315 extending from the curved partial-tubular shank 310, and a plurality of indentations 320 in the exterior surface the curved partial-tubular shank 310, into which bone can grow when the anchor 300 is inserted into a vertebra, locking the anchor 300 in place. The plurality of indentations 320 also provide resistance to motion when the anchor 300 is inserted into a vertebra, tending to hold the anchor in place. The curved partial-tubular shank 310 may have a cylindrical outer profile or a triangular, quadrilateral, or other polygonal outer profile. The straight tapered tip portion 315 may be tapered in a direction of an axis that is distinct from the axis of the straight tapered tip portion 315.

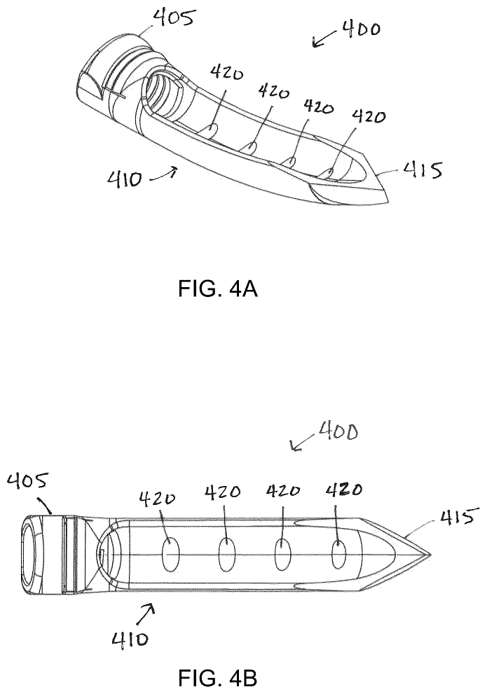

FIGS. 4A-4B show interior views of an embodiment of an anchor, including interior grooves. The anchor 400 includes a head 405, a curved partial-tubular shank 410 extending from the head 405, a straight tapered tip portion 415 extending from the curved partial-tubular shank 410, and a plurality of protrusions 420 on the interior surface the curved partial-tubular shank 410, in between which bone can grow when the anchor 400 is inserted into a vertebra, locking the anchor 400 in place. In another embodiment of an anchor, a curved partial-tubular shank 410 may comprise a plurality of grooves (not shown) in an interior of the curved partial-tubular shank 410, into which bone can grow when the anchor 400 is inserted into a vertebra, locking the anchor 400 in place. The plurality of grooves also provide resistance to motion when the anchor 400 is inserted into a vertebra, tending to hold the anchor in place. The curved partial-tubular shank 410 may have a cylindrical outer profile or a triangular, quadrilateral, or other polygonal outer profile. The straight tapered tip portion 415 may be tapered in a direction of an axis that is distinct from the axis of the straight tapered tip portion 415.

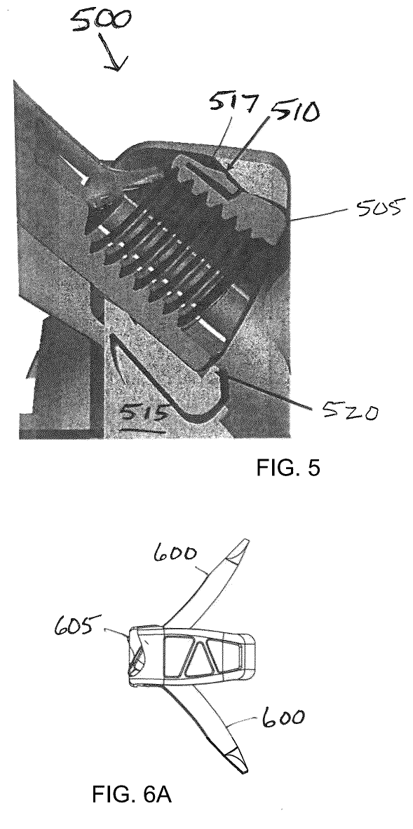

FIG. 5 shows a cross-section of a head of an embodiment of an anchor. The head 505 of the anchor 500 includes an anchor locking tab 510 flexibly affixed to a body of the head 505 that is depressed inward toward the head 505 as the anchor is inserted into the interbody 515 and which springs outward into a pocket 517 in an interbody receptacle for the head 505, thus engaging the pocket 517 and preventing motion of the head 505 out of the interbody 515. The interbody 515 also includes an interbody locking tab 520 that is moved outward while the head 505 is being inserted into the interbody 515 and to spring inward back once the head 505 is fully inserted, engaging the head 505 and preventing motion of the head 505 out of the interbody 515. Embodiments of the invention can include one or both of the anchor locking tab 510 and the interbody locking tab 520.

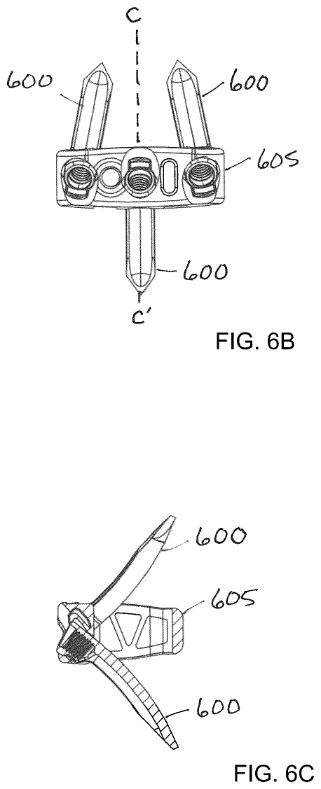

FIGS. 6A-6C show anchors and an interbody together. FIGS. 6A and 6B show exterior views of anchors 600 together with an interbody 605 with which the anchors 600 are designed to operate. FIG. 6C shows a cross-section through C-C′ of FIG. 6B.

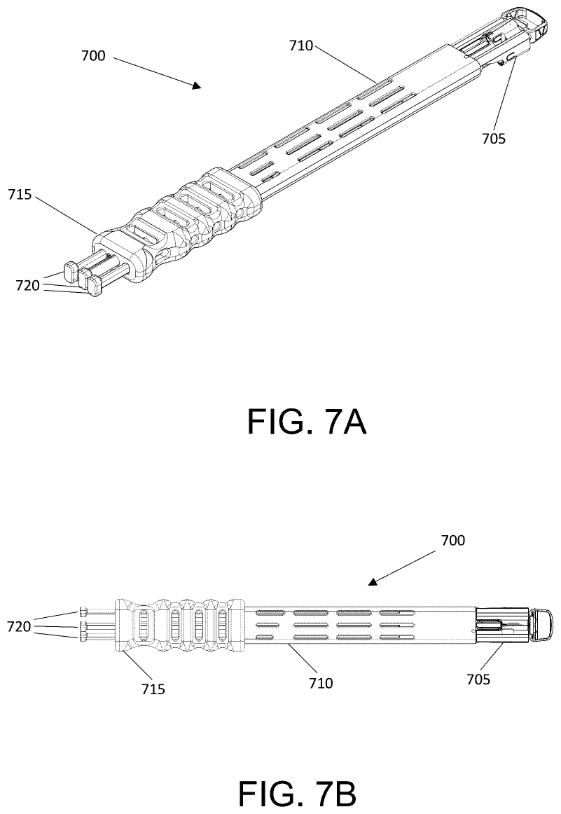

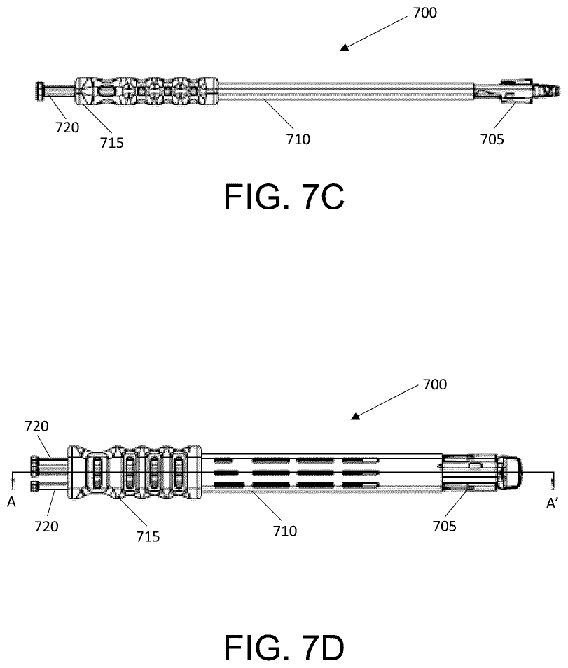

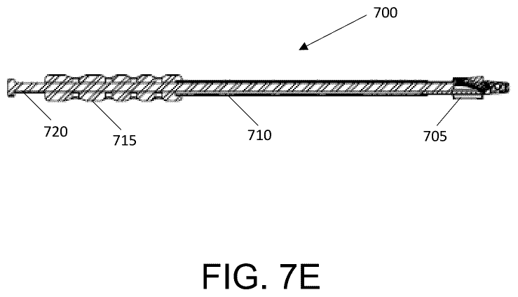

FIGS. 7A-7E show various views of an insertion tool 700 of an embodiment of the invention. FIG. 7A shows an orthogonal view of the insertion tool 700, which is used to manipulate/insert an interbody (e.g., 505) then manipulate an anchor (e.g., 100) for insertion into the interbody (e.g., 505). Tamp rods 720 are inserted through the insertion tool from a distal end 715 to translate impaction forces into the anchors, enabling the insertion of the anchors into the endplates of the vertebra. The insertion tool 700 includes a proximal end 705; a body 710, and a distal end 715. FIG. 7B shows a top view of the insertion tool 700, including the proximal end 705, the body 710, and the distal end 715. FIG. 7C shows a side view of the insertion tool 700, including the proximal end 705, the body 710, and the distal end 715. FIG. 7D shows a bottom view of the insertion tool 700, including the proximal end 705, the body 710, and the distal end 715. FIG. 7E shows a cross-section of the insertion tool 700 through A-A′ of FIG. 7D, including the proximal end 705, the body 710, and the distal end 715.

Embodiments of the present invention may include a curved partial-tubular shank (e.g., 110) with an interior that tapers from a head (e.g., 105) toward a straight tapered tip portion (e.g., 115), to create a thicker cross-section closer to the head and, when inserted into the interbody, closer to the interbody. Embodiments of the present invention my include a straight tapered tip portion (e.g., 115) with reliefs on the straight tapered tip portion. Embodiments of the present invention may also include a straight tapered tip portion (e.g., 115) extending from the curved partial-tubular shank (e.g., 105) non-tangentially from the curve of the curved partial-tubular portion. Embodiments of the present invention may also include a head (e.g., 105) that includes threads within the head for removal of an anchor (e.g., 100). Embodiments of the present invention may also include porous material in the interior of a curved partial-tubular shank (e.g., 105) for bone growth within the curved partial tube. Embodiments of the present invention may also include porosity in an external surface, an internal surface, or both, of a curved partial-tubular shank (e.g., 105).

As embodied and broadly described herein, an aspect of the present disclosure relates to an anchor and interbody for interbody fusion, comprising, consisting essentially of, or comprising: an interbody for insertion between vertebrae of a human spine, comprising: an interbody cage comprising a plurality of anchor receptacles, each anchor receptacle configured to receive an anchor to secure the interbody to a vertebra; and the anchor, comprising: a head configured to engage one of the plurality of anchor receptacles; a curved partial-tubular shank connected to or integral with the head at a proximal end of the curved partial-tubular shank, extending from the head, and comprising a plurality of concentric grooves on an exterior surface of the curved partial-tubular shank; and a straight tapered tip portion at a distal end of the curved partial-tubular shank and extending from the curved partial-tubular shank. In one aspect, the anchor further comprises a plurality of teeth disposed on an edge of the curved partial-tubular shank. In another aspect, the anchor further comprises a plurality of indentations in an exterior surface of the curved partial-tubular shank. In another aspect, the anchor further comprises a plurality of protrusions on an interior surface the curved partial-tubular shank. In another aspect, the anchor further comprises a plurality of grooves in an interior surface the curved partial-tubular shank. In another aspect, the anchor further comprises porosity in an external surface, an internal surface, or both, of the curved partial-tubular shank. In another aspect, an inner diameter and an outer diameter of the curved partial-tubular shank are not concentric, such that the curved partial-tubular shank varies in thickness. In another aspect, the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody. In another aspect, the interbody further comprises an interbody locking tab configured to be moved outward while the head is being inserted into the interbody and to spring inward once the head is fully inserted into the interbody, engaging the head. In another aspect, the straight tapered tip portion comprises one or more reliefs.

As embodied and broadly described herein, an aspect of the present disclosure relates to an anchor and interbody kit for interbody fusion, comprising, consisting essentially of, or comprising: an interbody for insertion between vertebrae of a human spine, comprising: an interbody cage comprising a plurality of anchor receptacles, each anchor receptacle configured to receive an anchor to secure the interbody to a vertebra; and the anchor, comprising: a head configured to engage one of the plurality of anchor receptacles; a curved partial-tubular shank connected to or integral with the head at a proximal end of the curved partial-tubular shank, extending from the head, and comprising a plurality of concentric grooves on an exterior surface of the curved partial-tubular shank; and a straight tapered tip portion at a distal end of the curved partial tubular shank and extending from the curved partial-tubular shank; and one or more tools configured to manipulate the interbody or the anchor. In one aspect, the anchor further comprises a plurality of teeth disposed on an edge of the curved partial-tubular shank. In another aspect, the anchor further comprises a plurality of indentations in an exterior surface of the curved partial-tubular shank. In another aspect, the anchor further comprises a plurality of protrusions on an interior surface the curved partial-tubular shank. In another aspect, the anchor further comprises a plurality of grooves in an interior surface the curved partial-tubular shank. In another aspect, the anchor further comprises porosity in an external surface, an internal surface, or both, of the curved partial-tubular shank. In another aspect, an inner diameter and an outer diameter of the curved partial-tubular shank are not concentric, such that the curved partial-tubular shank varies in thickness. In another aspect, the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody. In another aspect, the interbody further comprises an interbody locking tab configured to be moved outward while the head is being inserted into the interbody and to spring inward once the head is fully inserted into the interbody, engaging the head. In another aspect, the straight tapered tip portion comprises one or more reliefs.

As embodied and broadly described herein, an aspect of the present disclosure relates to a method for using an anchor and interbody for interbody fusion, consisting essentially of, or comprising: providing a patient in need of an interbody fusion; providing an anchor and interbody for interbody fusion, comprising: an interbody cage comprising a plurality of anchor receptacles, each anchor receptacle configured to receive an anchor to secure the interbody to a vertebra; and the anchor, comprising: a head configured to engage one of the plurality of anchor receptacles; a curved partial-tubular shank connected to or integral with the head at a proximal end of the curved partial-tubular shank, extending from the head, and comprising a plurality of or concentric grooves on an exterior surface of the curved partial-tubular shank; and a tapered tip portion at a distal end of the curved partial-tubular shank; inserting the interbody between two vertebrae of the patient; and inserting the anchor into one of the plurality of anchor receptacles. In one aspect, the anchor further comprises a plurality of teeth disposed on an edge of the curved partial-tubular shank. In another aspect, the anchor further comprises a plurality of indentations in an exterior surface of the curved partial-tubular shank. In another aspect, the anchor further comprises a plurality of protrusions on an interior surface the curved partial-tubular shank. In another aspect, the anchor further comprises a plurality of grooves in an interior surface the curved partial-tubular shank. In another aspect, the anchor further comprises porosity in an external surface, an internal surface, or both, of the curved partial-tubular shank. In another aspect, an inner diameter and an outer diameter of the curved partial-tubular shank are not concentric, such that the curved partial-tubular shank varies in thickness. In another aspect, the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody. In another aspect, the interbody further comprises an interbody locking tab configured to be moved outward while the head is being inserted into the interbody and to spring inward once the head is fully inserted into the interbody, engaging the head. In another aspect, the straight tapered tip portion comprises one or more reliefs.

While various features of the present invention are discussed and illustrated separately for clarity, each and every feature disclosed herein can be combined with any other disclosed feature in an embodiment unless such a combination in physically impossible.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property (ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112 (f) as it exists on the date of filing hereof unless the words “means for” or “step for” are explicitly used in the particular claim.

What is claimed is:

1. An anchor and interbody for interbody fusion, comprising:
an interbody for insertion between vertebrae of a human spine, comprising:
an interbody cage comprising a plurality of anchor receptacles, each anchor receptacle configured to receive an anchor to secure the interbody to a vertebra; and
the anchor, comprising:
a head configured to engage one of the plurality of anchor receptacles;
a curved partial-tubular shank connected to or integral with the head at a proximal end of the curved partial-tubular shank, extending from the head, and comprising a plurality of concentric grooves on an exterior surface of the curved partial-tubular shank; and
a straight tapered tip portion at a distal end of the curved partial-tubular shank and extending from the curved partial-tubular shank.

2. The anchor and interbody of claim 1, wherein the anchor further comprises a plurality of teeth disposed on an edge of the curved partial-tubular shank.

3. The anchor and interbody of claim 1, wherein the anchor further comprises a plurality of indentations in an exterior surface of the curved partial-tubular shank.

4. The anchor and interbody of claim 1, wherein the anchor further comprises a plurality of protrusions on an interior surface the curved partial-tubular shank.

5. The anchor and interbody of claim 1, wherein the anchor further comprises a plurality of grooves in an interior surface the curved partial-tubular shank.

6. The anchor and interbody of claim 1, wherein the anchor further comprises porosity in an external surface, an internal surface, or both, of the curved partial-tubular shank.

7. The anchor and interbody of claim 1, wherein an inner diameter and an outer diameter of the curved partial-tubular shank are not concentric, such that the curved partial-tubular shank varies in thickness.

8. The anchor and interbody of claim 1, wherein the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody.

9. The anchor and interbody of claim 1, wherein the interbody further comprises an interbody locking tab configured to be moved outward while the head is being inserted into the interbody and to spring inward once the head is fully inserted into the interbody, engaging the head.

10. The anchor and interbody of claim 1, wherein the straight tapered tip portion comprises one or more reliefs.

11. An anchor and interbody kit for interbody fusion, comprising:
an interbody for insertion between vertebrae of a human spine, comprising:
an interbody cage comprising a plurality of anchor receptacles, each anchor receptacle configured to receive an anchor to secure the interbody to a vertebra; and
the anchor, comprising:
a head configured to engage one of the plurality of anchor receptacles;
a curved partial-tubular shank connected to or integral with the head at a proximal end of the curved partial-tubular shank, extending from the head, and comprising a plurality of concentric grooves on an exterior surface of the curved partial-tubular shank; and
a straight tapered tip portion at a distal end of the curved partial tubular shank and extending from the curved partial-tubular shank; and
one or more tools configured to manipulate the interbody or the anchor.

12. The anchor and interbody kit of claim 11, wherein the anchor further comprises a plurality of teeth disposed on an edge of the curved partial-tubular shank.

13. The anchor and interbody kit of claim 11, wherein the anchor further comprises a plurality of indentations in an exterior surface of the curved partial-tubular shank.

14. The anchor and interbody kit of claim 11, wherein the anchor further comprises a plurality of protrusions on an interior surface the curved partial-tubular shank.

15. The anchor and interbody kit of claim 11, wherein the anchor further comprises a plurality of grooves in an interior surface the curved partial-tubular shank.

16. The anchor and interbody kit of claim 11, wherein the anchor further comprises porosity in an external surface, an internal surface, or both, of the curved partial-tubular shank.

17. The anchor and interbody of claim 11, wherein an inner diameter and an outer diameter of the curved partial-tubular shank are not concentric, such that the curved partial-tubular shank varies in thickness.

18. The anchor and interbody kit of claim 11, wherein the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody.

19. The anchor and interbody of kit claim 11, wherein the interbody further comprises an interbody locking tab configured to be moved outward while the head is being inserted into the interbody and to spring inward once the head is fully inserted into the interbody, engaging the head.

20. The anchor and interbody of kit claim 11, wherein the straight tapered tip portion comprises one or more reliefs.

21. A method for using an anchor and interbody for interbody fusion, comprising:
providing a patient in need of an interbody fusion;
providing an anchor and interbody for interbody fusion, comprising:
an interbody cage comprising a plurality of anchor receptacles, each anchor receptacle configured to receive an anchor to secure the interbody to a vertebra; and
the anchor, comprising:
a head configured to engage one of the plurality of anchor receptacles;
a curved partial-tubular shank connected to or integral with the head at a proximal end of the curved partial-tubular shank, extending from the head, and comprising a plurality of or concentric grooves on an exterior surface of the curved partial-tubular shank; and
a tapered tip portion at a distal end of the curved partial-tubular shank;
inserting the interbody between two vertebrae of the patient; and
inserting the anchor into one of the plurality of anchor receptacles.

22. The method of claim 21, wherein the anchor further comprises a plurality of teeth disposed on an edge of the curved partial-tubular shank.

23. The method of claim 21, wherein the anchor further comprises a plurality of indentations in an exterior surface of the curved partial-tubular shank.

24. The method of claim 21, wherein the anchor further comprises a plurality of protrusions on an interior surface the curved partial-tubular shank.

25. The method of claim 21, wherein the anchor further comprises a plurality of grooves in an interior surface the curved partial-tubular shank.

26. The method of claim 21, wherein the anchor further comprises porosity in an external surface, an internal surface, or both, of the curved partial-tubular shank.

27. The method of claim 21, wherein an inner diameter and an outer diameter of the curved partial-tubular shank are not concentric, such that the curved partial-tubular shank varies in thickness.

28. The method of claim 21, wherein the anchor further comprises an anchor locking tab configured to be depressed inward toward the head as the anchor is inserted into the interbody and the interbody further comprises a pocket configured to engage with the anchor locking tab when it springs outward once the anchor is inserted fully into the interbody.

29. The method of claim 21, wherein the interbody further comprises an interbody locking tab configured to be moved outward while the head is being inserted into the interbody and to spring inward once the head is fully inserted into the interbody, engaging the head.

30. The method of claim 21, wherein the straight tapered tip portion comprises one or more reliefs.
